(19) United States
(12) Patent Application Publication
(10) Pub. No.: US 2021/0055188 A1
(43) Pub. Date: Feb. 25, 2021

(54) METHODS FOR ISOLATING, DETECTING, AND/OR ANALYZING ANTIBODIES FROM A FORMALIN FIXED PARAFFIN EMBEDDED (FFPE) TISSUE SAMPLE

(71) Applicant: SHIMADZU CORPORATION, Kyoto (JP)

(72) Inventors: Noriko IWAMOTO, Kyoto (JP); Takashi SHIMADA, Kyoto (JP)

(21) Appl. No.: 16/939,572

(22) Filed: Jul. 27, 2020

Related U.S. Application Data
(60) Provisional application No. 62/888,794, filed on Aug. 19, 2019.

(51) Int. Cl. G01N 1/28; G01N 1/38; G01N 33/68

(52) U.S. Cl. CPC G01N 1/286; G01N 1/38; G01N 2001/385; G01N 2001/2866; G01N 33/6854

(57) ABSTRACT

Certain embodiments are directed to methods for preparing or processing FFPE samples for subsequent analysis of antibodies or other proteins contained within the FFPE sample. In certain aspects, preparing or processing a FFPE sample can include, but is not limited to (i) deparaffinizing a portion of the FFPE specimen, (ii) decrosslinking the deparaffinized sample portion, (iii) homogenizing the decrosslinked portion, and (iv) fractionating the homogenized portion, wherein isolated antibodies or proteins having a higher order structure that can be bound by capture agents, e.g., immunoglobulin capture agents.

### PRIORITY

The present application claims priority to U.S. Provisional Application No. 62/888,794 filed Aug. 19, 2019; the entire contents of which is incorporation herein by reference.

### STATEMENT REGARDING FEDERALLY FUNDED RESEARCH

None.

### BACKGROUND

For over a hundred years pathology clinics, laboratories, hospitals, and museums have been preserving biological specimens with formalin and other chemical fixatives such as formaldehyde and ethyl alcohol. The most common fixative is formalin. Formalin is used as a fixative because of its superior ability to preserve both tissue structure and cellular morphology. This has resulted in the wide use of formalin for the successful preservation of histologic sections for traditional microscopic analysis. Formalin fixation is so effective in preserving tissue structure and cellular morphology that formalin archives contain millions of samples. Within this archive are biological samples of healthy tissue, tissue samples from virtually every known human disease, and a multitude of preserved life forms.

Retrospective studies of tumors use samples that have been formalin fixed and paraffin embedded (FFPE). Analyses of various tumor types (breast, lung, prostate and colon) have revealed that there exist numerous subtypes of tumors within each anatomically defined cancer. Routine processing of samples in the clinical setting is significantly different from that conducted in a research laboratory. In particular, for routine analysis of biopsies from a clinical setting, the tissue is processed by formalin fixation and subsequently paraffin embedded. This process is a highly efficient method that is currently the standard in pathology suites.

Formalin fixation occurs through the crosslinking of proteins within the biological specimen. These protein crosslinks, while providing excellent cellular morphology preservation, also renders the fixed sample relatively insoluble. Because of these protein crosslinks, the types of assays that can be performed on a formalin-fixed sample are limited in number, unable to provide quantitative results and lack sensitivity. In fact, formalin fixed biological samples are virtually unusable in many modern assay techniques, which are both highly quantitative and sensitive.

There remains a need for additional methods of preparing or processing FFPE samples for subsequent analyses.

### SUMMARY

Certain embodiments are directed to methods for preparing or processing FFPE samples for subsequent analysis of antibodies or other proteins contained within the FFPE sample. In certain aspects, preparing or processing a FFPE sample can include, but is not limited to (i) deparaffinizing a portion of the FFPE specimen, (ii) decrosslinking the deparaffinized sample portion, (iii) homogenizing the decrosslinked portion, and (iv) fractionating the homogenized portion, wherein isolated antibodies or proteins have a higher order structure that can be bound by capture agents, e.g., immunoglobulin capture agents. As used herein the term “higher order structure” refers to the maintenance of secondary, tertiary, and/or quaternary protein structure, a portion of which is bound by a capture agent. Such higher order structures can specifically exclude unordered protein aggregates that fail to provide the appropriate three dimensional structure for analysis. A sample portion can include one or more slices or sections, or a core of a tissue that is FFPE. In certain embodiments, the sample is a FFPE specimen.

In certain aspects, deparaffinizing the sample portion can include extracting the sample portion with an organic solvent, such as a heptane/methanol solvent. Typically the extraction procedure is followed by drying the sample portion that remains, i.e., the non-paraffin portion not soluble in the organic solvent.

Decrosslinking the deparaffinized sample portion can be performed by incubating the deparaffinized sample portion in a buffered solution having a pH between 8.5 and 10 at a temperature between 50 and 65° C. for at least, at most, about, or for 6 to 48 hours forming a decrosslinked portion. The decrosslinked portion can be subjected to homogenization to form a homogenized portion. The homogenized portion can be fractionated. Fractionating a homogenized portion can separate the homogenized portion into a liquids fraction containing soluble proteins and a solids fraction. In certain aspects, the liquids fraction contains isolated antibodies or other proteins that have a higher order structure (e.g., secondary, tertiary, and/or quaternary structure) that can be bound by capture agents, such as immunoglobulin capture agents. In certain aspects, decrosslinking is performed at an alkaline pH. The alkaline pH can be about, at least, at most, or in the range of 8.5, 9.0, 9.5, to 10, including all values and ranges there between. In certain aspects, decrosslinking is performed at a pH of 9.5+/−0.5. A deparaffinized sample can be subjected to decrosslinking for 6, 8, 10, 12, 14, 16, 18, 20, 22, 24, 26, 28, 30, 32, 34, 36, 38, 40, 42, 44, 46 to 48 hours, including all values and ranges there between. In certain aspects, the sample portion is subjected to decrosslinking for 6 to 48 hours, more specifically 10 to 16 hours. In certain aspects, decrosslinking is carried out at a temperature of 50 to 60° C., including all values and ranges there between. The decrosslinking buffer solution can be a tris(hydroxymethyl)aminomethane hydrochloride (Tris-HCl) solution. In certain aspects, the buffered solution can include 10 to 100 mM buffer component. In certain aspects, the buffered solution can be a 20 mM Tris-HCl solution.

Homogenization of the decrosslinked sample portion can be performed using a variety of methods to disrupt the tissue and release polypeptide components. In certain aspects, homogenization can be open homogenization or closed homogenization. Closed homogenization can be performed using a closed homogenization apparatus. In certain aspects, homogenization of the decrosslinked sample portion can be performed by homogenization by grinding with homogenization beads. Homogenization can be performed in an appropriate homogenization buffer. In certain aspects, a homogenization buffer can containing 0.5 to 1.5 weight percent nonionic detergent. In certain aspects, the homogenization buffer contains octylthioglucoside (OTG). In certain aspects the homogenization buffer can contain 0.25, 0.5, to 1 weight percent (wt %) OTG.

In certain aspects, the antibody is an biologic drug. The antibody can be a monoclonal antibody. In certain aspects, the monoclonal antibody can be nivolumab, pembrolizumab, atezolizumab, avelumab, durvalumab, tremelimumab, adalimumab, bevacizumab, rituximab, panitumumab, ofatumumab, golimumab, ipilimumab, tocilizumab, trastuzumab, trastuzumab-DM1, omalizumab, mepolizumab, gemtuzumab ozogamicin, palivizumab, ramucilimab, siltuximab, obiltoxaximab, mogamulizumab, eculizumab, cetuximab, infliximab, and/or basiliximab.

The specimen can be a tissue biopsy. In certain aspects, the specimen can be a tumor biopsy. In a particular instance the tumor biopsy can be from a patient administered a therapeutic antibody.

In certain embodiments, the method can further include analyzing antibodies isolated from the specimen. In certain aspects, analyzing the isolated antibodies includes conducting mass spectrometry analysis of the isolated antibodies or fragments thereof. The mass spectrometry analysis can be, but is not limited to liquid chromatography mass spectrometry (LCMS). In a particular aspect, the antibody can be analyzed by nano-surface and molecular-orientation limited (nSMOL) proteolysis.

The methods can further include (i) diluting the liquid fraction of the homogenized sample portion with a buffered detergent solution forming a diluted sample portion; (ii) contacting the diluted sample portion with an immunoglobulin capture agent; (iii) eluting immunoglobulin bound to the immunoglobulin capture agent; (iv) immobilizing the eluted immunoglobulin in a reaction pore; (v) contacting the reaction pore with beads chemically modified with a protease, e.g., trypsin (trypsin beads), the beads having a larger diameter than the reaction pore; (vi) incubating the reaction pore in contact with beads for 4-6 hours at 50° C. forming a limited peptide digest containing peptides; and (vii) collecting peptides and subjecting the collected peptides to analysis by mass spectrometry. The detergent solution can be a neutral detergent solution. In certain aspects, the neutral detergent can be octylthioglycoside (OTG). In a further aspect the detergent solution can be a 0.1% octylthioglycoside (OTG) solution. The mass spectrometry can be LCMS.

“Antibodies” are usually heterotetrameric glycoproteins of about 150,000 daltons, composed of two identical light (L) chains and two identical-heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain.

The term “variable” refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) or hypervariable regions both in the light chain and the heavy chain variable domains. The more highly conserved portions of variable domains are called the framework (FR). The variable domains of heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab)2 fragment that has two antigen combining sites and is still capable of cross-linking antigen.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab″ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group; F(ab′)2 antibody fragments originally were produced as pairs of Fab′ fragments which have hinge cysteines between them. Other, chemical couplings of antibody fragments are also known.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and μ, respectively. The subunit structures and three-dimensional configurations of different classes of immunoglobulins are well known.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, scFv and Fv), so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains,” “containing,” “characterized by,” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The patent or application file contains at least one drawing executed in color. Copies of this patent or patent application publication with color drawing(s) will be provided by the Office upon request and payment of the necessary fee. The invention may be better understood by reference to one or more of these drawings in combination with the detailed description of the specification embodiments presented herein.

FIG. 1. Scheme of antibody drug measurement from FFPE Samples.

FIG. 2. Illustration of conditions for binding of IgG to Protein A beads. The IgG was incubated at the indicated Detergent (1%) and temperature and then reacted with Protein A beads. A time-dependent increase in binding of IgG to protein A was confirmed, but incubation with 1% SDS was performed IgG lost its ability to bind to Protein A beads.

FIG. 3. Decrosslinking Condition Considerations for FFPE Samples. The FFPE cell block of SKBR3 reacted with Trastuzumab is reacted overnight at 60° C. in a buffer of various pH from 20 mM Tris-HCl pH 4.0 to pH 10.0, and crush with beads, and after extracting protein, measurement of Trastuzumab bound to HER2 of SKBR3 was performed by nSMOL-LCMS method. The FFPE cell block of A431 reacted with cetuximab is reacted as the same condition of SKBR3 block.

FIG. 4. Decrosslinking Condition Considerations for FFPE Samples. React SKBR3 reacted with Trastuzumab and react A431 reacted with cetuximab FFPE cell block overnight in a buffer of 20 mM to 300 mM Tris-HCl (pH 9.5) at 60° C., and crush with beads, and after extracting protein, the Trastuzumab binding to HER2 of SKBR and Cetuximab binding to EGFR of A431 were measured by nSMOL-LCMS method.

FIG. 5. Investigation of detergent for protein extraction from FFPE samples. React SKBR3 reacted with Trastuzumab and react A431 reacted with cetuximab FFPE cell block overnight in a buffer of 20 mM Tris-HCl (pH 9.5) at 60° C., and crush with beads, and after extracting protein by the noted detergent, Trastuzumab and Cetuximab were measured by nSMOL-LCMS method.

FIG. 6. Detergent concentration optimization for protein extraction from FFPE samples. React SKBR3 reacted with Trastuzumab and react A431 reacted with cetuximab FFPE cell block overnight in a buffer of 20 mM Tris-HCl (pH 9.5) at 60° C., and crush with beads, the examination was conducted at the concentration of OTG at 0.25% to 1% (final concentration). Similar examinations were made for OG, which is similar in structure to OTG, but the result was different from OTG.

FIG. 7. Impact of formalin fixation time of FFPE samples on nSMOL-LCMS detection. After collecting cells of A431 cells reacted with Cetuximab and collecting cells of SKBR3 reacted with Trastuzumab by centrifugation, the formalin fixation time in making the FFPE cell block was examined for 1 to 4 days. After decrosslink reaction, bead crushing, and protein extraction, each antibody was measured by nSMOL-LCMS.

FIG. 8. Detection of Trastuzumab from FFPE cancer tissue of mouse human cancer xenograft model. Administration of Trastuzumab in a xenograft model in which human breast cancer cells BT-474 were transplanted in mice and detection Trastuzumab accumulated in cancer tissues derived from BT-474 derived tumor tissue was conducted. The dosage of Trastuzumab for Group 3 (20 mg/kg) was doubled from the concentration of Group 2 (10 mg/kg). The antibody concentration was twice as high in Group 3 (3F-1, 3F-2, 3F-3) than in Group 2 (2F-1, 2F-2. 2F-3). When measured, Trastuzumab accumulated in cancer tissue by nSMOL-LCMS method.

FIG. 9. Detection of Trastuzumab from FFPE Tissues of Mouse Human Cancer Xenograft Model. Trastuzumab amount was detected in tissues (lung, heart, liver, spleen, kidney, pancreas, cancer tissue) of mice treated with Trastuzumab in a xenograft model transplanted with human breast cancer cell BT-474 in mice by the method of the present invention, it was confirmed that Trastuzumab is accumulated specifically in cancer tissues.

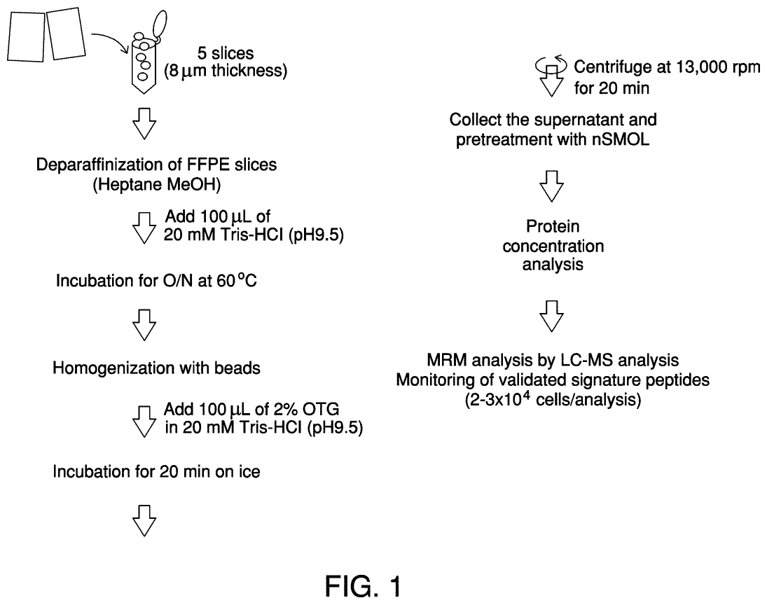

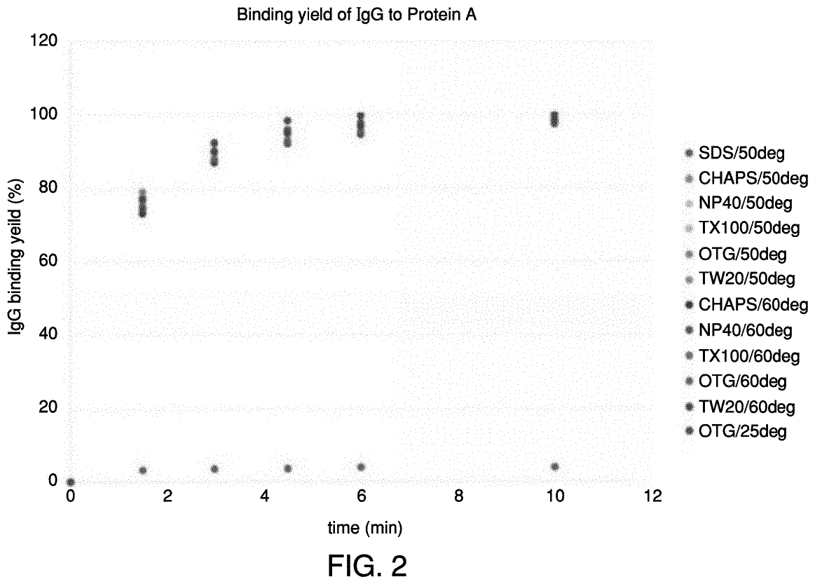

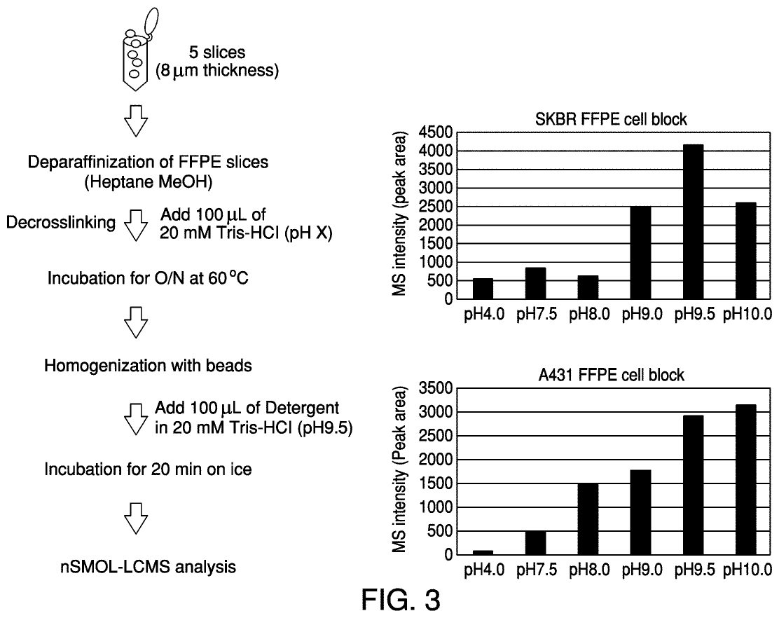

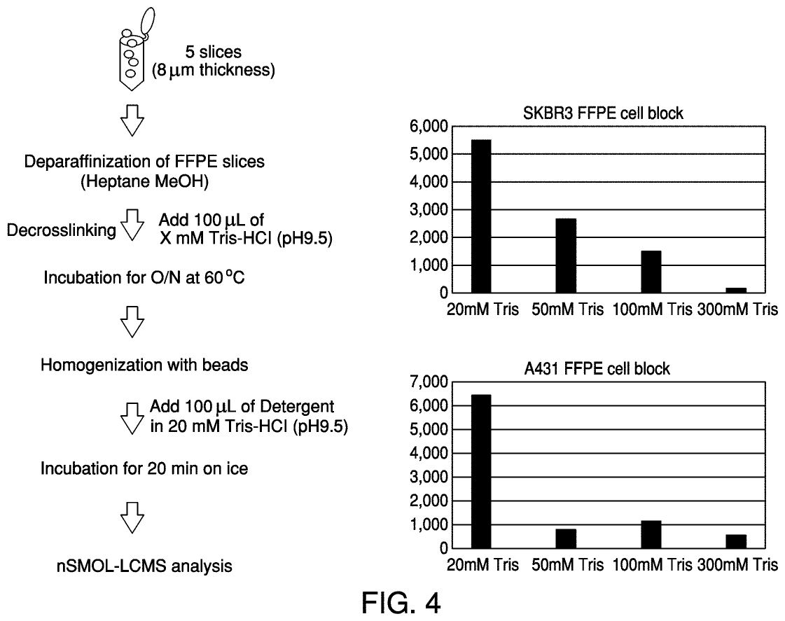

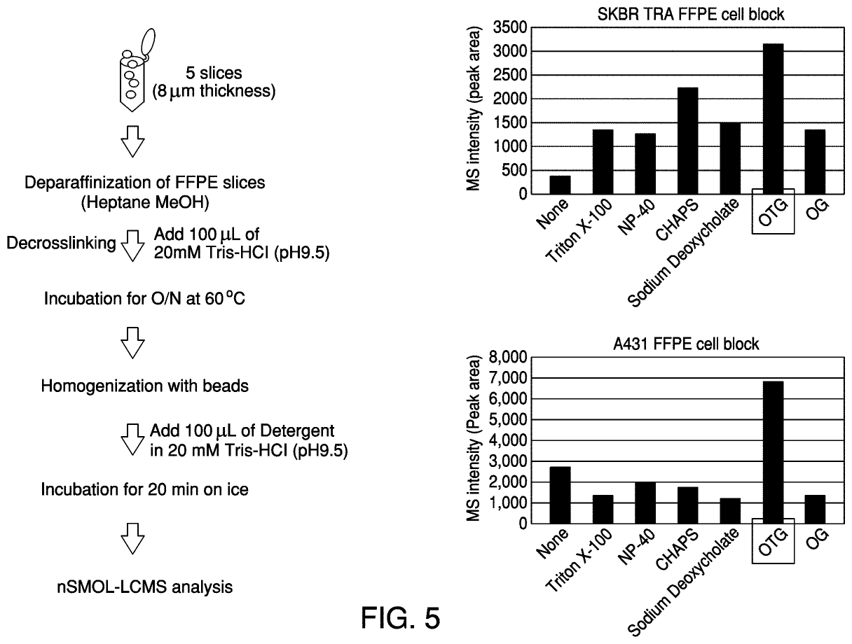

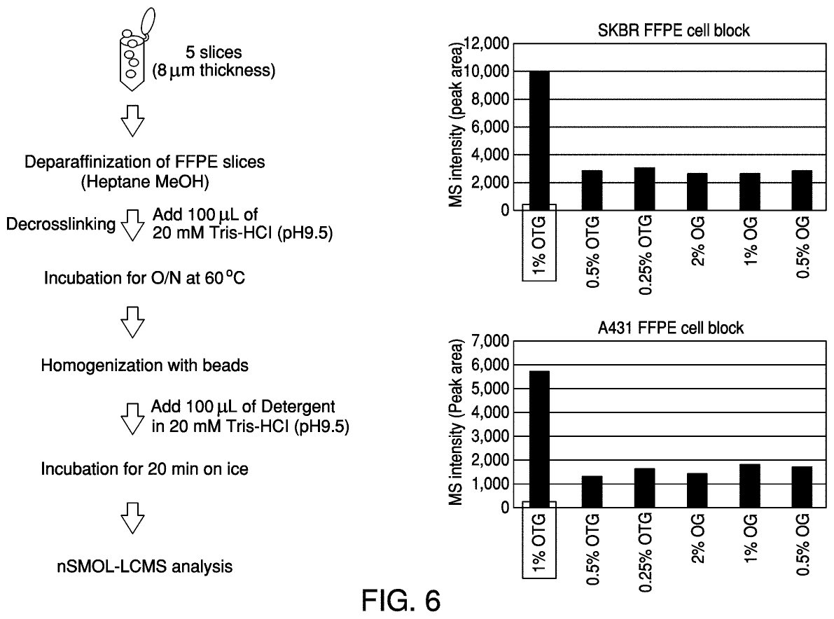

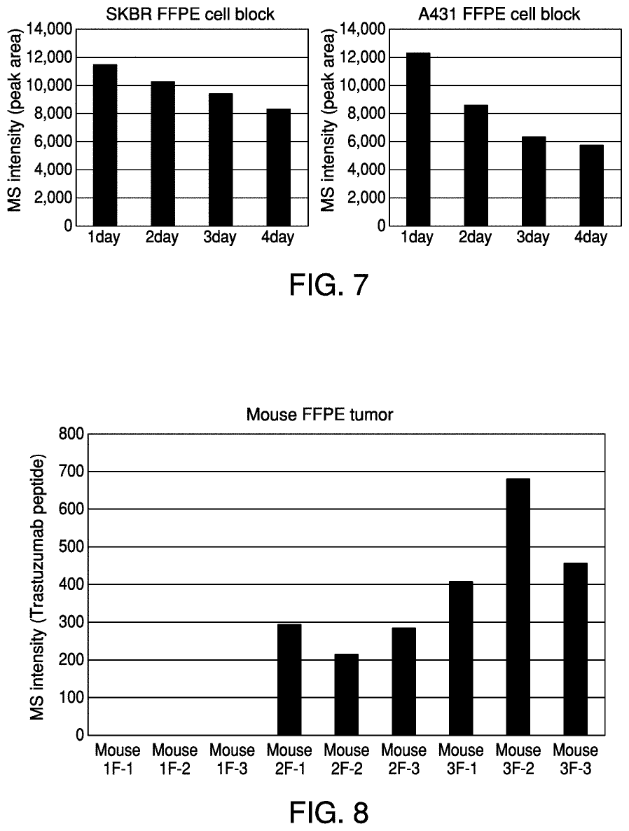

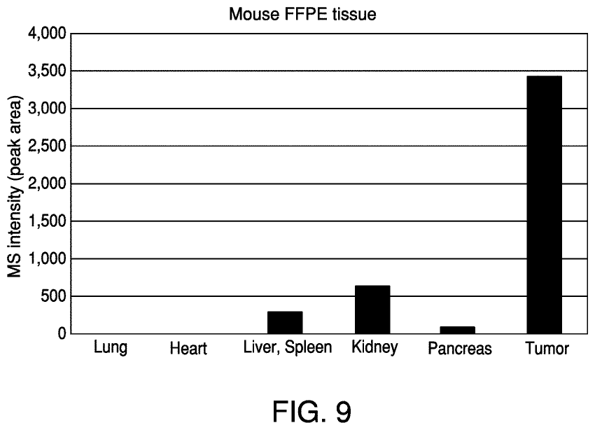

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain embodiments are directed to methods for analysis of FFPE samples. In certain aspects, the methods provide for the preparing or processing of a FFPE sample for further analysis. In certain aspects, preparing or processing a FFPE sample can include, but is not limited to (i) deparaffinizing a portion of the FFPE specimen, (ii) decrosslinking the deparaffinized sample portion, (iii) homogenizing the decrosslinked portion, and (iv) fractionating the homogenized portion, wherein isolated antibodies or proteins having a higher order structure that can be bound by capture agents, e.g., immunoglobulin capture agents.

### I. Preparation or Processing of Paraffin-Embedded Specimen

FFPE samples or specimens can be used in a variety of analyses including transcriptome, proteome, morphologic, immunohistochemical, and enzyme-histochemical analyses. Certain embodiments described herein are directed to preparing or processing FFPE specimens for analysis using limited proteolysis and peptide identification. A portion of a FFPE specimen can be used, e.g., one or more slices or a core of a FFPE sample. Typically, paraffin is used as embedding media for such specimens.

One example of a paraffin-embedded specimen is prepared by using a petroleum-based paraffin wax alone as an embedding medium. Here, the petroleum-based wax refers to a mixture of hydrocarbons that are derived from petroleum and are solid at normal temperature. Further, the term hydrocarbons normally means saturated hydrocarbons having a molecular weight of about 300 to 500 which comprises linear hydrocarbons (normal paraffins) with an average carbon number of about 20 to 35 as a main component.

Another example of a paraffin-embedded specimen is a specimen prepared by using an embedding medium containing the above-described petroleum-based paraffin wax as a base and further containing additional ingredients. As such additional ingredients, any ingredient that may be added for the purpose of improving quality of the embedding medium or the like are accepted. As an example of an embedding medium in which additional ingredients can be blended includes (i) embedding medium in which a petroleum-based microcrystalline wax, polyisobutylene, an ethylene-vinyl acetate copolymer and polybutene are blended as the additional ingredients for the purpose of improving workability in slicing and cracking resistance at low temperature; (ii) embedding medium in which polyisobutylene, an ethylene-vinyl acetate copolymer and saturated fatty acid are blended as the additional ingredients for the purpose of lowering the melting point, and improving workability in slicing and cracking resistance at low temperature, and the like. Specimens for analysis according to the methods described herein can be embedded in any paraffin based embedding medium. The paraffin embedding medium is not limited to any particular medium.

Deparaffinization. In certain aspects, a FFPE specimen or a portion thereof is deparaffinized, i.e., the paraffin embedding medium is removed or extracted from the specimen leaving the fixed tissue substantially free of embedding medium. As used herein substantially free of embedding medium includes a quantity of residual paraffin of less than, about, or of 10, 5, 1, 0.5, 0.1 weight percent (w %) of the dried deparaffinized sample. In certain aspects, the paraffin embedded specimen or portion thereof is subjected to an organic solvent that is compatible with embedding medium. The solvent dissolves the paraffin embedding medium under a normal temperature conditions. Typically, this operation can be repeated numerous times. An organic solvent that is compatible with paraffin (solubilizes paraffin) can be used. An example of organic solvent in the present invention may be selected from heptane, xylene, chloroform, diethyl ether, lemosol, and alcohols (for example, methanol, isopropyl alcohol and the like). These organic solvents may be used individually or in a mixture of two or more solvents. In certain aspects, the extraction solvent can be a heptane/methanol solvent. In certain aspects the extraction solvent can be 50:1, 40:1, 30:1, 25:1, 20:1, 15:1, 10:1, 5:1 organic solvent/alcohol (e.g., heptane/methanol) including all values and ratios there between.

Deparaffinization can include the step of exposing a sample/specimen embedded in paraffin to an organic solvent(s) that are compatible with the paraffin, to melt and dissolve the paraffin in the organic solvent; and removing the paraffin from the specimen by separating the organic solvent containing dissolved paraffin, from the exposed sample. The above two steps may be conducted stepwise, or at once. After deparaffinization further washing may be conducted by immersing and retaining in an organic solvent (with no previously dissolved paraffin, that is fresh solvent). In certain aspects, the specimen, the organic solvent, or the specimen and the solvent can be processed at room temperature (20, 21, 22, 23, 24, to 25° C.) or heated to 40, 45, 50, 55, to 60° C. In certain aspects, the sample and solvent can be incubated for at most, at least, about, or for 5, 6, 7, 8, 9, 10, 15, to 20 minutes. The incubation can be repeated 1, 2, 3, 4, 5, 6, or more times. Once paraffin is removed from the paraffin-embedded specimen the crosslinked tissue is exposed. The exposed sample can then be dried, for example, with the remaining residual solvent being removed by evaporation or the like. Once deparaffinized the sample can be subjected to decrosslinking procedure or process.

### II. Decrosslinking

Protein, e.g., antibody, isolation from formaldehyde-fixed tissues is hindered by the crosslinking or bond formation between the amino groups of proteins and formaldehyde. Decrosslinking involves the reversal of these protein/formaldehyde cross-links. Typically the deparaffinized sample can be incubated in an appropriate buffer at an appropriate pH, at an appropriate temperature, for an appropriate time to effect decrosslinking.

In certain aspects the decrosslinking step of the methods described herein can be performed in an aqueous solution at an alkaline pH. The alkaline pH can be about, at least, at most, or in the range of 8.5, 9.0, 9.5, to 10, including all values and ranges there between. In certain aspects, decrosslinking is performed at a pH of 9.5+/−0.5. Decrosslinking components can be performed or incubated for 6, 8, 10, 12, 14, 16, 18, 20, 22, 24, 26, 28, 30, 32, 34, 36, 38, 40, 42, 44, 46 to 48 hours, including all values and ranges there between. In certain aspects, the sample portion is subjected to decrosslinking for 6, 8, 10, 12, 14, 16, 18, 20, 22, 24, 26, 28, 30, 32, 34, 36, 38, 40, 42, 44, 46, to 48 hours, more specifically 10 to 16 hours. In certain aspects, decrosslinking is carried out at a temperature of 50, 51, 52, 53, 54, 55, 56, 57, 58, 59, to 60° C., including all values and ranges there between. In certain aspects, the buffer compound can contain a primary amine (e.g., tris(hydroxymethyl)aminomethane (Tris) buffer), and the buffer solution having a pH of between 4 and 10, including all values and ranges there between. The decrosslinking buffered solution can be a tris(hydroxymethyl)aminomethane hydrochloride (Tris-HCl) solution. In certain aspects, the buffered solution can include 10, 20, 30, 40, 50, 60, 70, 80, 90, to 100 mM buffer component. In certain aspects, the buffered solution can be a 20 mM Tris-HCl solution.

### II. Homogenization

Homogenizing the decrosslinked sample portion can be performed using a variety of methods to disrupt the tissue and release polypeptide components, e.g., samples can be homogenized by any standard mechanical, sonic, or other suitable technique. Tissue homogenization is preferably carried out with homogenization beads. Bead beating is an effective mechanical method used to disrupt a wide range of biological samples. Typically, bead beating is accomplished by rapidly agitating a sample with grinding media (beads or balls) in a device that shakes the homogenization vessel, i.e., a bead beater. Bead beaters have been designed to homogenize samples in microwell plates, tubes, or vials with beads or balls that are made of glass (silica), ceramic (zirconium) or stainless steel. Samples can be processed with or without buffer or solvent at either ambient or cryogenic temperatures. A variety of devices can be used for bead homogenization (bead beating). A tube, vial, or plate is shaken so that grinding media can impact and disrupt the sample. Vortexers, the simplest (and least effective) bead beater, work by swirling the sample and beads/balls in a motion that promotes disruption. Other bead beaters, including dental amalgamators and shaking mills, oscillate tubes, often in a figure-eight motion, which allows for crushing and grinding of samples. High throughput homogenizers, which can process samples in deep well plates in addition to other formats, have a linear motion that focuses the kinetic energy of the grinding media on the sample instead of on the sides of the container. In addition, a number of commercially available homogenizers are suitable for use with the invention including: Ultra-Turrax homogenizer (IKA-Works, Inc., Wilmington, N.C.); Tissumizer (Tekmar-Dohrmann, Cincinnati, Ohio); and Polytron (Brinkmann, Westbury, N.Y.).

In certain aspects, homogenizing can be open homogenization or closed homogenization. Closed homogenization can be performed using a closed homogenization apparatus. In certain aspects homogenizing the decrosslinked sample portion can be performed by homogenization by grinding with homogenization beads. Homogenization can be performed in an appropriate homogenization solution/buffer.

In one embodiment, the sample is homogenized in the presence of a homogenization solution containing a detergent agent. In certain aspects, the detergent is a nonionic detergent. A detergent can be selected such that at an effective concentration proteins, e.g., antibodies, are isolated from processed sample in an amount greater than that isolated in the absence of the detergent. The detergent can be a mild detergent that does not denature proteins, for example, a nonionic or amphoteric ionic detergent. Such detergents include octylthioglucoside (OTG), digitonin, polyoxyethylene alkylether (Brij series), polyoxyethylene sorbitan (Tween series), 0-dodecylmaltoside, β-octylglucoside, β-nonylglucoside, β-heptylglucoside, sucrose mono-decanoate, sucrosemonododecanoate, octyltetraoxyethylene, octylpentaoxyethylene, dodecyloctaoxyethylene, N,N-dimethyldecylamine-N-oxide, N,N-dimethyldodecylamine-N-oxide, N,N-dimethyldodecylammonio propanesulfonate, octyl (hydroxyethyl)sulfoxide, octanoyl-N-methylglucamide, nonanoyl-N-methylglucamide, decanoyl-N-methylglucamide, and (3-[(3-cholamidepropyl)-dimethylammonio]-1-propanesulfonate (CHAPS). In certain aspects, a detergent includes or is OTG. The homogenization solution can be buffered to a pH of 7.5 to 10, with a suitable biochemical buffer such as Tris-HCl. In certain aspects, the homogenization solution contains protease inhibitors. Representative examples of protease inhibitors include, but are not limited to: serine protease inhibitors (such as phenylmethylsulfonyl fluoride (PMSF), benzamide, benzamidine HCl, F-Amino-n-caproic acid and aprotinin (Trasylol)); cysteine protease inhibitors, such as sodium p-hydroxymercuribenzoate; competitive protease inhibitors, such as antipain and leupeptin; covalent protease inhibitors, such as iodoacetate and N-ethylmaleimide; aspartate (acidic) protease inhibitors, such as pepstatin and diazoacetylnorleucine methyl ester (DAN); metalloprotease inhibitors, such as EGTA [ethylene glycol bis(β-aminoethyl ether) N,N,N′,N′-tetraacetic acid], and the chelator 1, 10-phenanthroline.

In certain aspects, a homogenization solution can contain 0.5, 1.0, 1.5, to 2 weight percent nonionic detergent. In certain aspects the homogenization solution contains octylthioglucoside (OTG). In certain aspects, the homogenization buffer can contain 0.25, 0.5, 1.0, 1.5, to 2 weight percent (wt %) OTG.

### IV. Fractionation

The homogenized sample can be fractionated to isolate the soluble protein fraction of the homogenized sample. Typically, a soluble protein fraction is obtained. In certain aspects, the homogenate is centrifuged or filtered to remove particulate debris. The soluble protein fractions are then separated. In certain aspects, a protein concentration filter (e.g., AMICON or Millipore PELLICON ultrafiltration units) may be used. In certain embodiments, an antibody may then be purified from the soluble protein fraction. An antibody can be isolated or purified from other soluble proteins and polypeptides by immunoaffinity.

### V. Analysis and Detection of Antibodies

Therapeutic antibodies typically have a pathogenic protein as a target antigen and are currently attracting attention as a molecularly targeted medicine. Since antibodies are proteins present in the body, it is expected that side effects and adverse event will be limited, and the antibody can be administered at a sufficient concentration to provide a therapeutic effect. Antibodies have high molecular specificity and can be accumulated in a target lesion. In characterizing the effectiveness of a therapeutic antibody it is important to assess an optimum dosage by measuring the localization and concentration of the therapeutic antibody.

The most common technique for quantification of proteins such as antibodies is an enzyme linked immune-sorbent assay (ELISA). This is a technique for quantifying a target molecule by making an antibody against a target to be measured and sandwiching the antibody with a detection antibody. However, in an ELISA, for example, the target is not directly measured and it is necessary to prepare an antibody specific for each target and biological matrix for competitive processes. In particular, with regard to an antibody medicine, a cross reaction with an endogenous antibody can also occur resulting in inaccurate measurements. Furthermore, in a state where the neutralizing antibody and the antigen are bound, the antigen recognition site can be blocked and cannot be measured by ELISA.

In certain instances, when a target protein to be quantified is not associated with a commercial or detection antibody it is necessary to purify the protein in a large amount or use mass spectrometry. In processing a sample for immunohistochemistry the sample can be compromised and it is often difficult to determine a positive, false positive, and a negative result. One can identify the target protein in a specimen by using mass spectrometry. Current methods can be used to isolate certain cells of the specimen for analysis, e.g., laser dissection. For example, only cancer cells can be recovered, and expression variation analysis of target proteins in the cancer cells can be observed by mass spectrometry. When a protein in a sample is detected by mass spectrometry the protein is subjected to protease digestion producing peptide fragments. It is important to efficiently select a target peptide fragment from a variety of peptide fragments.

In certain aspects, the antibodies isolated using the methods described herein can be detected and/or analyzed by mass spectrometry. In certain embodiments, detection and analysis can be by nano-surface molecular orientation limited (nSMOL) proteolysis, see PCT publication WO 2016/143224, which is incorporated herein by reference. The isolated antibodies can be selectively cleaved and the resulting peptide measured and/or characterized. In the case of an antibody, it is necessary to selectively digest the antigen binding fragment (Fab domain), particularly the variable regions of a Fab domain, and to suppress digestion of the crystallization region (Fc domain).

An antibody target can be characterized by immobilizing the target antibody on a substrate and selectively digesting the antibody with a protease enzyme. In certain aspects, a monoclonal antibody to be measured is immobilized in a pore and nanoparticles having an immobilized protease are brought into contact with the pore containing the immobilized antibody to perform selective protease digestion of the monoclonal antibody. Selective protease digestion produces peptide fragments that are detected by a liquid chromatograph mass spectrometry (LCMS).

In certain aspects, selective protease digestion can include immobilizing a target antibody in a porous body and contacting the porous body containing the immobilized antibody in a pore of the porous body with nanoparticles having an immobilized protease on the surface of the nanoparticles. The dimensions of the nanoparticle are larger than the pore dimension providing for a physically limited digestion of the immobilized antibody. Selective or limited protease digestion of the monoclonal antibody results in peptide fragment that can be analyzed by LCMS. In certain aspects the average pore diameter of the porous body is in a range of 10 nm to 200 nm and the average particle size of the nanoparticles is larger than the average pore diameter of the porous body. Peptide fragments to be analyzed have an amino acid sequence derived from an antibody binding region of an antibody heavy chain or a light chain, e.g., the complementarity determining region (CDR). In certain aspect, the heavy chain or a light chain is a monoclonal antibody heavy chain or light chain.

The “CDR region” is a region having a particularly large amino acid substitution frequency in an antibody. A peptide detection target can be identified using the CDR region as an index. In certain aspects, a CDR peptide can be used for sequence alignment analysis.

Mass Spectrometry. Quantitative techniques using mass spectrometry are mainly performed by a hybrid type mass spectrometer called a triple quadrupole. Specifically, the ionized biomolecules first pass through a portion called octopole to reduce its ionic molecular vibration radius. Then, in the first quadrupole, ions having a specific mass number are selected by resonating, and other ions are excluded. This step is also referred to as single ion monitoring. The selected ion is carried to the second quadrupole, and cleavage is performed by collision with argon. This reaction is referred to as collision-induced dissociation. As a result of the cleavage reaction, the generated specific fragment is selected by the third quadrupole, very high sensitivity and high selective quantification can be performed. This series of analyses is referred to as multiple reaction monitoring. When connected to a high-speed liquid chromatograph, continuous analysis can be made possible.

In order to detect an antibody by mass spectrometry, it is necessary to extract an antibody from a biological sample such as blood or tissue and dissolve the antibody in an appropriate solvent. In addition, since the antibody has a large molecule to be analyzed as it is, the antibody is decomposed into a peptide by a protease and then subjected to mass analysis after being separated by a liquid chromatograph. The molecular weight of the peptide suitable for analysis is about 800 to 3,000 da. However, when a general protein molecule is protease-decomposed, about 100 peptide fragments are generated from one protein, and in the case of an antibody, more than 200 peptide fragments are produced. In the antibody molecule, only a portion of the sequence (CDRs) varies from antibody to antibody, with the remaining portion having common sequences.

Certain embodiments are directed to methods of identifying the presence of and measuring the amount of a monoclonal antibody in a sample. A monoclonal antibody is immunoglobulin (IgG) of two heavy chains and two light chains connected by disulfide bonds. The heavy and light chains comprise a constant region and a variable region. The antibody framework has an amino acid sequence that is common in most antibodies of the same type. The variable region includes a complementarity determining region (CDR) comprising three CDR regions (CDR1, CDR2, CDR3) that form a three-dimensional structure that is involved in specific binding with the antigen.

In certain aspects, the CDR region of a monoclonal antibody can be selectively protease-digested and the identification and quantification of the antibody can be performed by mass analysis of the obtained peptide fragment. The analysis method includes detecting a peptide fragment derived from a variable region of an antibody, especially a fragment of a CDR region, the antibody is detected and quantified, and the peptide fragment derived from the antibody is directly measured. The method of the present invention is applicable regardless of the type of antibody, the present invention is not limited to the examples enumerated herein.

The porous body comprising the antibody capture agent is not limited as long as it has a large number of pores. Activated carbon, porous membrane, porous resin beads, metal particles, and the like can be used.

1. A method for isolating an antibody present in a formalin fixed paraffin embedded (FFPE) specimen comprising:
(i) deparaffinizing a portion of the FFPE specimen to form a deparaffinzed sample portion;
(ii) decrosslinking the deparaffinized sample portion by incubating the deparaffinized sample portion in a buffered solution having a pH between 8.5 and 10 at a temperature less than 65° C. for at least 6 hours to forma decrosslinked portion;
(iii) homogenizing the decrosslinked portion to form a homogenized portion;
(iv) fractionating the homogenized portion into a liquids fraction and a solids fraction, wherein the liquids fraction contains isolated antibodies having a higher order structure that can be bound by immunoglobulin capture agents; and
(v) analyzing the isolated antibodies using liquid chromatography mass spectrometry (LCMS).

2. The method of claim 1, wherein the deparaffinizing of the sample portion comprises extracting the sample portion with a heptane/methanol solution.

3. The method of claim 1, wherein the decrosslinking is performed at a pH of approximately 9.5.

4. The method of claim 1, wherein the decrosslinking comprises a 6 to 48 hour incubation.

5. The method of claim 4, wherein the decrosslinking incubation incubated for 10 to 16 hours.

6. The method of claim 1, wherein the decrosslinking is performed at a temperature between 50 to 60° C.

7. The method of claim 1, wherein the buffered solution is a tris(hydroxymethyl)aminomethane hydrochloride (Tris-HCl) solution.

8. The method of claim 7, wherein the Tris-HCl solution is a Tris-HCl solution having a concentration of approximately 20 mM.

9. The method of claim 1, wherein homogenizing the decrosslinked sample portion is performed in a closed homogenization apparatus.

10. The method of claim 1, wherein homogenizing the decrosslinked sample portion comprises homogenization by grinding with homogenization beads.

11. The method of claim 1, wherein homogenization is performed in a homogenization buffer containing 0.5 to 1.5 weight percent neutral detergent.

12. The method of claim 11, wherein homogenization is performed in a homogenization buffer containing octylthioglucoside (OTG).

13. The method of claim 12, wherein homogenization is performed in a homogenization buffer containing approximately 1 weight percent (wt %) OTG.

14. The method of claim 1, wherein the antibody is a biologic drug.

15. The method of claim 1, wherein the antibody is a monoclonal antibody.

16. The method of claim 15, wherein the monoclonal antibody is nivolumab, pembrolizumab, atezolizumab, avelumab, durvalumab, tremelimumab, adalimumab, bevacizumab, rituximab, panitumumab, ofatumumab, golimumab, ipilimumab, tocilizumab, trastuzumab, trastuzumab-DM1, omalizumab, mepolizumab, gemtuzumab ozogamicin, palivizumab, ramucilimab, siltuximab, obiltoxaximab, mogamulizumab, eculizumab, cetuximab, infliximab, or basiliximab.

17. The method of claim 1, wherein the specimen is a tissue biopsy.

18. The method of claim 1, wherein the specimen is a tumor biopsy.

19. The method of claim 1, further comprising:
(i) diluting the liquid fraction of the homogenized sample portion with a buffered detergent solution forming a diluted sample portion;
(ii) contacting the diluted sample portion with an immunoglobulin capture agent;
(iii) eluting immunoglobulin bound to the immunoglobulin capture agent;
(iv) immobilizing the eluted immunoglobulin in a reaction pore;
(v) contacting the reaction pore with beads chemically modified with trypsin (trypsin beads), the beads having a larger diameter than the reaction pore;
(vi) incubation the reaction pore in contact with trypsin beads for 4-6 hours at 50° C. forming a limited peptide digestion containing peptides; and
(vii) collecting peptides and subjecting the collected peptides to analysis by mass spectrometry.

20. The method of claim 19, wherein the detergent solution is a neutral detergent solution.

21. The method of claim 20, wherein the neutral detergent octylthioglycoside (OTG).

22. The method of claim 19, wherein the detergent solution is a octylthioglycoside (OTG) solution having a concentration of approximately 0.1%.
